(19) United States
(12) Patent Application Publication
(10) Pub. No.: US 2017/0361312 A1
(43) Pub. Date: Dec. 21, 2017

(54) ENGINEERED INERT MEDIA FOR USE IN FIXED BED DEHYDROGENATION REACTORS

(71) Applicant: SABIC Global Technologies B.V., Begen op Zoom (NL)

(72) Inventors: Guillermo Leal, Riyadh (SA); Khalid El-Yahyaoui, Riyadh (SA); Zeeshan Nawaz, Riyadh (SA)

(21) Appl. No.: 15/536,338

(22) PCT Filed: Dec. 15, 2015
(86) PCT No.: PCT/IB2015/059633

§ 371 Date: Jun. 15, 2017

Related U.S. Application Data
(60) Provisional application No. 62/092,462, filed on Dec. 16, 2014.

(51) Int. Cl. B01J 35/02; B01J 35/04; B01J 8/02; C07C 5/333; C07C 11/02

(52) U.S. Cl. CPC B01J 35/026; B01J 35/023; C07C 5/333; C07C 11/02; B01J 8/025; B01J 35/04; B01J 2219/30475; B01J 2219/30223

(57) ABSTRACT

A method of carrying out the dehydrogenation of lower alkanes in a fixed-bed reactor containing a catalyst bed, the catalyst bed comprising (i) catalyst particles supporting a catalyst effective to promote said dehydrogenation and (ii) engineered inert diluent particles, where the method comprises passing the lower alkane in gaseous form through the catalyst bed, wherein the engineered inert diluent particles have a cross-sectional shape having two opposing convex edges joined by and intersecting two opposing concave edges, and a plurality of holes between said edges penetrating through the particle.

### BACKGROUND

In recent years, increased demand for light olefin petrochemicals, particularly propylene and isobutylene, has led to the development of single step alkane dehydrogenation processes such as the CATOFIN™, Oleflex™, FBD-3 (for propylene) and FBD-4 (for isobutylene) processes. Such dehydrogenation processes convert isobutane to isobutene or propane to propylene over a supported catalyst, such as a chromia-alumina catalyst (CATOFIN® process) or a platinum catalyst (Oleflex® process) or any other catalyst that is able to dehydrogenate alkanes.

Briefly, such processes employ a series of fixed-bed reactors that undergo a controlled sequence of reaction and reheat/regeneration. At the start of the reaction (e.g., dehydrogenation) step, feed streams are vaporized, raised to the reaction temperature, and contacted with catalyst in a fixed bed. Because the reaction is endothermic, the reactor temperature drops during the reaction step. The reaction step is followed by a reheat/regeneration step, to prepare the catalyst bed for the next reaction phase. Typically, during the reheat step, any carbon (e.g., coke) deposited on the catalyst is also burned off. Conventionally, the regenerating (i.e., heating) gas is fed in the same direction as the process gas.

The catalyst beds of these reactors, which comprise catalyst on porous non-uniform inert support particles, are designed to provide suitable contact of feed to the catalyst surface and to accumulate and transfer heat during the endothermic dehydrogenation reaction.

Issues that can arise during such reactions include unfavorable temperature distributions and poor flow distribution within the catalyst bed and/or increased pressure drop across the reactor. Specifically, it may be observed during the reaction cycle that the bottom temperature of the catalyst bed is substantially lower than the top temperature, the difference sometimes reaching 100° C. or more. Such an unfavorable temperature profile can lead not only to unsatisfactory catalyst utilization and cracking but also to undesirable side reactions.

Increases in pressure drop can be caused by deposition of feed contaminants or corrosion products on or within the catalyst particles, and/or by breakdown of catalyst particles, leading to crusting or agglomeration. This can cause channeling of reagent gas within the bed, leading to uneven reaction rates, and reduce evenness and efficiency of heat transfer, thus contributing to uneven temperature distribution.

Other complications include loss of catalyst due to attrition, which is aggravated by physical contact between catalyst particles, and sintering, i.e., the loss of catalyst surface activity due to crystal growth of either the support material or the active catalyst phase. Loss of active catalyst can lead to an increase in unwanted side reactions. In addition, one of the many disadvantages associated with using inert particles that are non-uniform is that attrition of such inert particles increases with space velocity. This attrition leads to increased production of fine particles (“fines”) due to the abrasion and/or other physical interactions between the inert particles and the catalyst particles. The production of fines increases the pressure drop across the catalyst bed, which adversely affects both the reactor bed heat sink distribution of the catalyst bed, as well as the flow distribution. When these effects occur in a dehydrogenation reactor, the yield of alkenes is reduced.

The use of graded beds, where catalyst particles are diluted with inert diluent particles, with a higher void content at the top of the reactor, has been shown to improve (reduce) pressure drop. An example of a graded bed is shown in FIG. 1, where hot gas (e.g., steam or combustion gas) 10 moves through three beds at T₀ and warm gas 12 leaves the beds at T₃. Overall length, L, of the grade bed is 400 millimeters (mm) where various mixtures of a cylindrical catalyst 2 having a length of 3.2 mm and spherical inert packing 4, 6, 8 are present in beds 14, 16, 18. Spherical inert packing 4 has a length of 6.4 mm. Spherical inert packing 6 has a length of 9.5 mm. Spherical inert packing 8 has a length of 12.7 mm. However, further improvements in the design of fixed catalyst beds are desirable to achieve optimum heat sink effect and pressure drop, minimum attrition, improved flow distribution and maximum yield of alkenes from alkanes.

### SUMMARY

Disclosed in various embodiments are engineered inert particles, methods of making engineered inert particles, and a catalyst beds in a fixed-bed reactor utilizing engineered inert particles.

An engineered inert particle for use as a diluent in a catalyst bed in a fixed-bed reactor, comprises: an engineered inert particle comprising a cross-sectional shape having two opposing convex edges joined by and intersecting two opposing concave edges and a plurality of holes between said edges penetrating through the particle.

A catalyst bed in a fixed-bed reactor for use in dehydrogenation of lower alkanes, comprises: (i) catalyst particles supporting a catalyst effective to promote said dehydrogenation; and (ii) engineered inert diluent particles comprising a cross-sectional shape having two opposing convex edges joined by and intersecting two opposing concave edges and a plurality of holes between said edges penetrating through the particle.

A method of carrying out the dehydrogenation of lower alkanes in a fixed-bed reactor containing a catalyst bed, the catalyst bed comprising (i) catalyst particles supporting a catalyst effective to promote said dehydrogenation and (ii) engineered inert diluent particles, the method comprises: passing the lower alkane in gaseous form through the catalyst bed, wherein the engineered inert diluent particles have a cross-sectional shape having two opposing convex edges joined by and intersecting two opposing concave edges, and a plurality of holes between said edges penetrating through the particle.

These and other features and characteristics are more particularly described below.

### BRIEF DESCRIPTION OF THE DRAWINGS

The following is a brief description of the drawings wherein like elements are numbered alike and which are presented for the purposes of illustrating the exemplary embodiments disclosed herein and not for the purposes of limiting the same.

FIG. 1 shows an example of a graded catalyst bed, where catalyst particles are diluted with spherical inert diluent particles, with a higher void content at the top of the reactor.

FIG. 2 is a two-dimensional representation of catalyst particles mixed with engineered inert particles, in a fixed bed, where arrows represent gas flow through the bed.

FIG. 3 is a two-dimensional representation of catalyst particles mixed with inert diluent particles without defined shape in a fixed bed, where arrows represent gas flow through the bed, and regions of “channeling” are circled.

FIG. 4 is a schematic diagram of the experimental apparatus used to determine the different parameters to determine the benefits of the engineered media versus the non-engineered media for catalyst beds.

FIG. 5 is a graph illustrating the effect on pressure drop within a fixed bed reactor of (i) substitution of inert diluents particles as disclosed herein for prior art inert diluent particles and (ii) changes in weight ratio of diluents particles to catalyst particles.

FIG. 6 is a graph illustrating the effect on temperature gradient within a fixed bed reactor of (i) substitution of inert diluents particles as disclosed herein for prior art inert diluents particles and (ii) changes in weight ratio of diluents particles to catalyst particles.

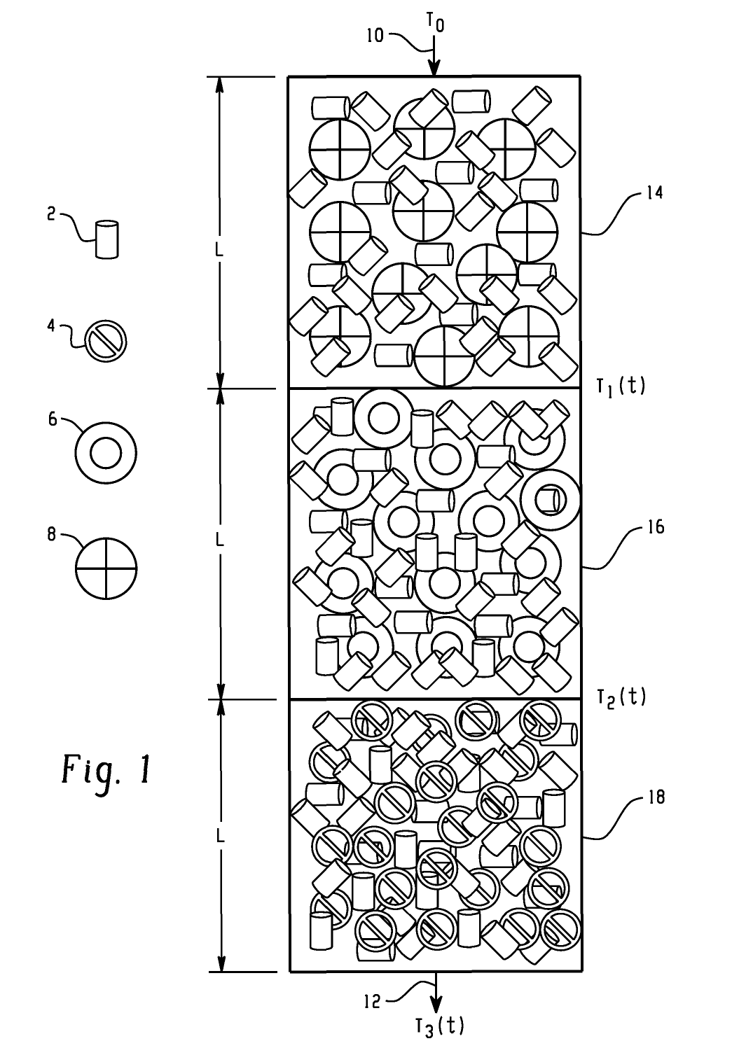

### DETAILED DESCRIPTION

Disclosed herein is an engineered inert media for use in catalytic reactors, non-limiting examples of which include fixed bed reactors used to dehydrogenate alkanes. Also disclosed herein are methods for using such engineering inert media in catalytic reactions.

As disclosed herein, engineered inert particles, also referred to herein as engineered inert packing particles and engineered inert diluent particles, can be used as a diluent in a catalyst bed. The engineered inert particles can be used with any reactor, for example the engineered inert particles can be used in fixed-bed reactors, such as, for example, fixed-bed dehydrogenation reactors, in which the engineered inert particles can enhance catalyst bed heat sink capacity, reduce catalyst bed temperature gradients, decrease pressure drop, increase flow distribution and reduce attrition, thus improving the utilization and performance of the catalyst. Optionally, the engineered inert packing particles can be used with existing dehydrogenation process, non-limiting examples of which include CATOFIN™, Oleflex™, FBD-3 (Snamprogetti/Yarsintez process, for propylene), and FBD-4 (for isobutylene) processes, but they can also be used in other alkane dehydrogenation processes as well as other processes utilizing fixed-bed heterogeneous catalysis. The process can comprise an endothermic reaction, of which alkane dehydrogenation is an example. The engineered inert particles, as described herein, can be used as the sole inert diluent in a particular catalyst bed, or combined with other inert diluents. Also contemplated by the engineered inert particles and processes disclosed herein can be the use of combinations of different engineered and non-engineered inert particles in the same catalyst bed.

The shape of the engineered inert diluent particle determines the ratio of external surface to volume of the particle and can play an important role in the heat and mass transfer of the mixed catalyst/diluent bed. The inert diluent disclosed herein can thus improve pressure drop performance, flow distribution, heat sink capacity, and temperature distribution within the catalyst bed.

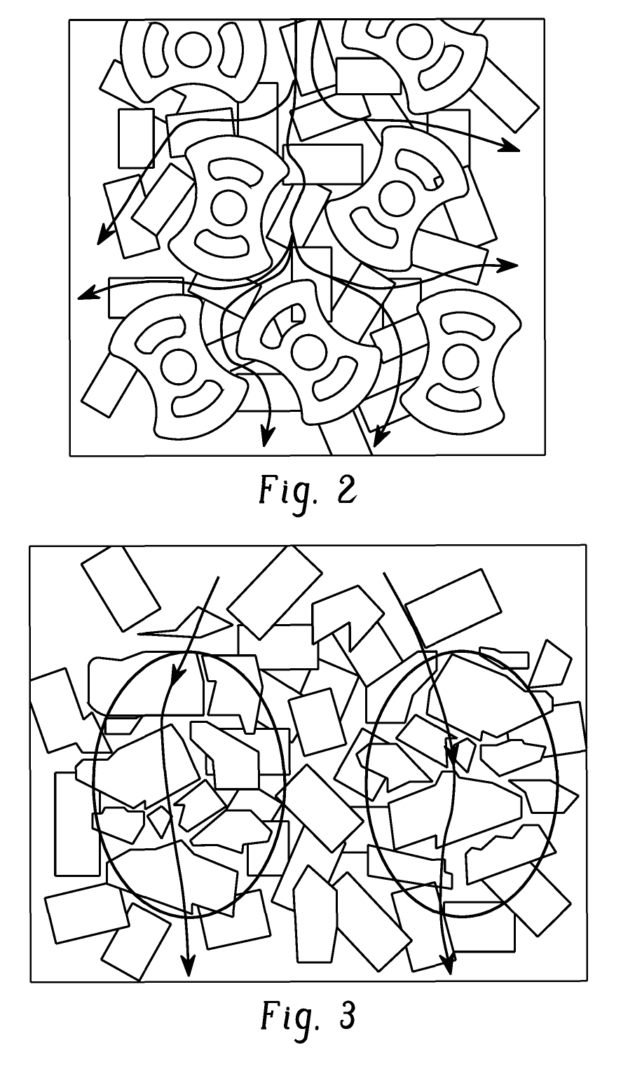

In general, the engineered inert particle can have a cross-sectional shape having two opposing convex edges joined by and intersecting two opposing concave edges, and a plurality of holes between the edges that penetrate through the particle. Optionally, the two opposing convex edges can have substantially the same radius of curvature. Likewise, optionally, the two opposing concave edges can have substantially the same radius of curvature. Additionally, each of the edges of the cross-sectional shape can optionally have substantially the same radius of curvature. As referred to herein, “substantially the same radius of curvature” generally refers to the radius of curvatures being compared as being nearly equivalent. For example, one radius of curvature can have a value that is greater than or equal to a value of the other radius of curvature, for example, greater than or equal to 50%. For example, one radius of curvature can have a value that is greater than or equal to 75%, for example, greater than or equal to 85%, for example, greater than or equal to 90%, for example, greater than or equal to 95%, for example, greater than or equal to 99%, for example, greater than or equal to 99.5% of a value of the other radius of curvature. An exemplary cross-sectional shape is illustrated in FIG. 2. The corners at which the convex and concave edges “intersect” can be pointed, rounded, or slightly rounded and, in most cases, do not form protrusions or lobes. As shown in FIG. 2, the corner at which the convex and concave edges “intersect” can be slight rounded. Optionally, the radius of curvature of the convex edges of the particle cross-section is equal to that of a circle having a diameter equal to the length of the particle. The concave edges can also have this radius of curvature, as shown, for example, in FIG. 2.

The size of the inert particles can vary, but generally the particles can have a length (i.e., longest dimension, from the midpoint of one edge to the midpoint of the opposing edge on the same surface) of greater than or equal to 5 millimeters (mm), for example, greater than or equal to 6 mm, for example, greater than or equal to 7 mm, for example, greater than or equal to 8 mm, for example, greater than or equal to 9 mm, for example, greater than or equal to 10 mm, for example, greater than or equal to 15 mm. For example, the length can be greater than or equal to 6.4 mm. The length can be less than or equal to 40 mm, for example, less than or equal to 38 mm, for example, less than or equal to 35 mm, for example, less than or equal to 30 mm, for example, less than or equal to 25 mm, for example, less than or equal to 20 mm, for example, less than or equal to 15 mm, for example, less than or equal to 10 mm. The length of the inert particles can be 5 mm to 40 mm, 10 mm to 30 mm, or 15 mm to 25 mm. The particles can have a thickness (perpendicular to the plane of the cross section defined above) that is generally between 25% to 100% of the length of the particle; e.g., 25%, 30%, 40%, 50%, 75%, or 100% of the length of the particle.

The holes that penetrate the inert particle can, optionally, be cylindrical or substantially cylindrical (e.g., having a cross section that is not circular) and can connect opposite faces of the particle. Note that, in this context, the word “holes” refers to macroscopic features of the particle shape and not to the porosity of the material from which the particle is made. When the holes are cylindrical or substantially cylindrical, the holes optionally can be arranged such that the longitudinal axes of the holes are all parallel. As referred to herein, “substantially cylindrical” generally refers to an inert particle that is nearly cylindrical. For example, the inert particle having a substantially cylindrical shape can have a shape that is greater than or equal to 50% cylindrical. For example, the substantially cylindrical shape can be greater than or equal to 75%, for example, greater than or equal to 85%, for example, greater than or equal to 90%, for example, greater than or equal to 95%, for example, greater than or equal to 99%, for example, greater than or equal to 99.5%. A given inert particle can have one hole or a plurality of holes, such as, for example, two, three, or four holes. It will be understood that when the inert particle is fabricated with a plurality of holes, the holes need not have the same size or shape. For example, the plurality of holes can comprise, or consist of, a central hole and two holes which can define curved slots that are substantially parallel to the two convex edges (see e.g., FIG. 2). As referred to herein, “substantially parallel” generally refers to the curved slots being nearly parallel to the two convex edges. For example, the curved slots can be greater than or equal to 50% parallel to the two convex edges. For example, the curved slots can be greater than or equal to 75%, for example, greater than or equal to 85%, for example, greater than or equal to 90%, for example, greater than or equal to 95%, for example, greater than or equal to 99%, for example, greater than or equal to 99.5% parallel to the two convex edges. If desired, however, the plurality of holes may have the same size and/or shape. The surface area defined by the one or more cylindrical or substantially cylindrical holes may be equivalent to 10% to 60%, 15% to 50%, 20% to 45%, or 30% to 40% of the total cross sectional area defined by the intersecting edges described previously.

The size of the holes and/or the number of holes can be selected to optimize mass transfer and/or heat transfer for a particular reaction. Variation in the number of holes in an inert particle or the hole diameter of one or more holes of an inert particle can change the fluid dynamics of the reactant flow in the reactor. The number of holes can improve mass transfer until an optimum target is reached. These numbers of holes can have the main objective to achieve the best surface contact, decrease in pressure drop, and improve flow distribution together with heat sink capacity to maximize catalyst performance. The number and/or size of the holes can be chosen to maximize the yield of a dehydrogenation reaction in a fixed bed reactor. For example, the number of holes in the inert particle can be 1 to 10, for example, 2 to 8, or, for example, 3 to 7. For example, the inert particle can have 2, 3, 4, 5, or 6 holes. The size of each hole in a given inert particle can be described by its respective area. For example, the hole size can be 1 square mm (mm²) to 400 mm,² for example, 50 mm² to 350 mm², for example, 100 mm² to 300 mm², or, for example, 150 mm² to 250 mm².

Generally, the engineered inert diluent particles can be composed of materials that are inert under reaction conditions and resistant to high temperatures and to mechanical crushing. For example, the inert particles can be porous ceramic materials. Some non-limiting examples of materials for fabricating the inert particles can include, but are not limited to, alumina, silica, titania, magnesia, zirconia, metal carbides, silicon carbide, carbon and zeolites, or a combination comprising at least one of the foregoing. When the engineered inert particles are used in connection with alkane dehydrogenation to produce alkenes, such particles can often be made of alumina.

Also disclosed are reactors containing catalyst beds with the engineered inert particles as described herein. Optionally, the engineered inert particles in a given catalyst bed can be present in particular ratios relative to catalyst particles. When this occurs, the ratio of the amount of engineered inert particles to the amount of catalyst particles can be selected based upon the reaction under consideration and the operating conditions of the reactor used to run the reaction. For example, a catalyst bed in a fixed-bed reactor for use in dehydrogenation of lower alkanes can comprise: (i) catalyst particles supporting a catalyst, for example, a catalyst effective to promote such a dehydrogenation reaction, and (ii) engineered inert diluent particles as described herein. The weight ratio of inert diluent particles to catalyst particles can be greater than 1; for example, the weight ratio of inert diluent particles to catalyst particles can be at least 65:35. For example, the weight ratio of inert diluent particles to catalyst particles can be at least 1.5 to 1, at least 1.65 to 1, at least 1.75 to 1, at least 1.9 to 1, or at least 1.95 to 1. Optionally, the weight ratio of inert diluent particles to catalyst particles can be 2:1 or less.

If desired, the catalyst particles can be selected to be a different shape than the inert diluent particles. For example, the catalyst particles can be cylindrical, spherical, Raschig rings, or any other shape. The catalyst particles can be either hollow or solid, and the catalyst support materials can be porous.

The diluent particles can be large enough relative to the catalyst particles to facilitate the separation of the catalyst particles from the diluent particles, and the diluent particles from each other, in a packed bed. For example, the catalyst particles can be cylinders x mm in diameter, and the inert diluent particles can be larger than 2x mm in length. For example, the catalyst particles can be cylinders 3.2 mm in diameter and 4.4 mm in height, and the inert diluent particles can be larger than 6.4 mm in length. For example, the diluent particles can be less than 15 mm, for example, less than 10 mm in length.

A method for carrying out the dehydrogenation of lower alkanes in a fixed-bed reactor containing a catalyst bed, can include passing the lower alkane in gaseous form through the catalyst bed, where the catalyst bed can comprise: (i) catalyst particles supporting a catalyst effective to promote the dehydrogenation reaction and (ii) inert diluent particles as described herein.

The disclosed inert diluents and their use can provide significant benefits in dehydrogenation reactors, such as CATOFIN™ reactors, and similar fixed bed and plug flow reactors. The increase in surface area, together with the reduction of attrition and channeling, as discussed below, can provide benefits such as:
Reduction in formation of hot spots;
Improved flow distribution;
Improved temperature profile in the catalyst bed;
Reduction of secondary reactions in the bed, minimizing unwanted secondary products;
Decrease in the ΔP of the reactor, permitting increased flow;
Improved kinetics, due to increased surface area; and
Reduction of unplanned and planned shutdowns; increase in production.

The following example are merely illustrative of the device disclosed herein and are not intended to limit the scope hereof.

### EXAMPLES

### Example 1

The benefits of the disclosed engineered inert diluent particles and their use in fixed bed dehydrogenation reactions were demonstrated as described in the following example.

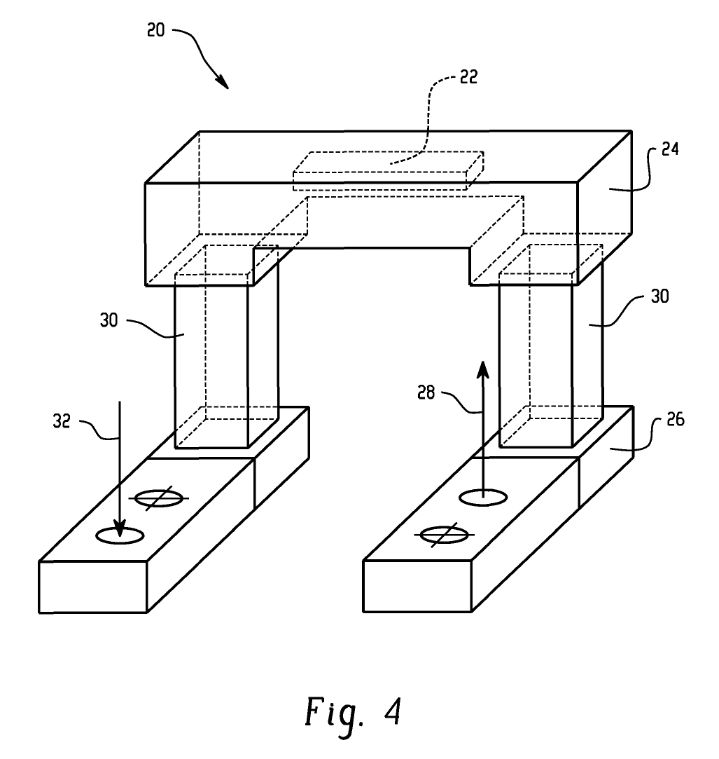

The inert packing characteristics of the engineered inert diluent particles were evaluated by means of packed bed heat transfer experiments, which were used to generate transient cooling and heating curves pertaining to representative locations inside the catalyst bed. The schematic diagrams for the packed-bed heating and cooling experiments are shown in FIG. 4. A packed tower 20 was used to conduct the experiments. In the packed tower 20, burner 22 is located in the top press tap 24. Test columns 30, having a height of 183 centimeters (cm) connected the top press tap 24 to the bottom press tap 26. Air inlet 32 and air outlet 28 are present in the bottom press tap 26. The experiments were carried out in this packed tower 20 having an inside diameter of 0.15 meter (m) or larger, to avoid any unwanted excessive heat loss. The packed-bed height was adjustable in the range of 1.0 m to 1.5 m. The cooling and heating experiments were conducted in the temperature range of 500-700° C. under vacuum, i.e. at a pressure of 0.3 to 0.5 atmosphere (30 to 50 kiloPascals (kPa).

The engineered inert particles were mixed with alumina catalyst pellets that were 3.2 mm in diameter and 4.4 mm in height. The heating and cooling medium was superheated (dry) steam, although combustion product gas from the natural gas/oil burner or hot air may also be used.

Pressure drop and temperature profiles using the disclosed inert diluent particles were compared with that obtained using T-64 particles for Comparative Example A (CEA). These alumina unshaped particles are available commercially in the open market which are irregular in shape (see FIG. 3). The production of solid fines due to abrasion between the catalyst particles and the inert particles is commonly observed in commercial dehydrogenation reactors.

For the same inlet flow rate, a pressure drop reduction of at least 19% resulted from the substitution of the FIG. 2 diluents for the T-64 grain at a 1:1 (wt %) catalyst:diluents ratio.

In addition, pressure drop and temperature profiles using the disclosed engineered support and hold down media, RSM-L and RSM-S were compared with that obtained using conventional inert spherical particles. The engineered support and hold down media, RSM-L and RSM-S, resulted in a significant pressure drop advantage of over 50% when compared to such conventional inert spherical particles. During tests conducted on the complete reactor system, utilization of the engineered support media, hold down media and diluents resulted in a significant pressure drop reduction of at least 12% when compared to the current reactor system used.

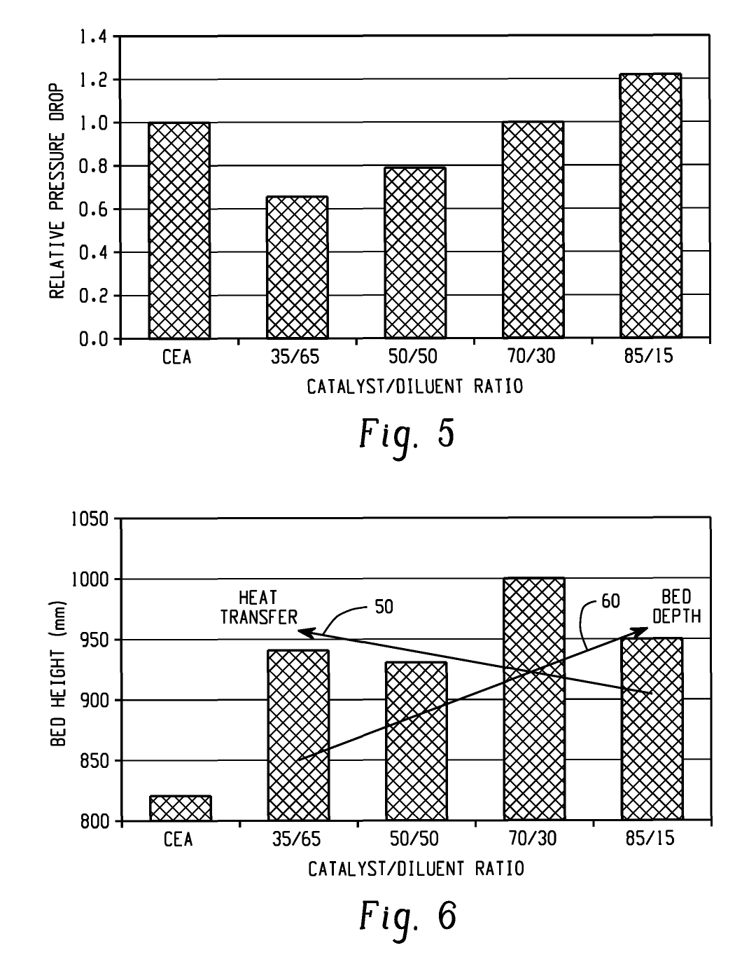

It was also shown that the pressure drop across the reactor was reduced as the ratio of catalyst to diluent was decreased. As shown in FIG. 5, relative pressure drop was reduced to as low as 65% of the 1:1 system at a catalyst/diluent ratio of 35/65 and increased to 122% at a catalyst/diluent ratio of 85/15. Comparative Example A (CEA) used the T-64 particles.

The temperature profile in the catalyst bed from top to bottom was also significantly improved, as shown in FIG. 6, when the disclosed inert diluent was utilized. FIG. 6 illustrates the bed height from a top to a bottom of the bed when exposed to temperatures greater than or equal to 600° C. The height of the bed maintained above a temperature of 600° C. increased by an average of about 16% relative to the T-64 system (50/50 wt % catalyst/diluent ratio). The increase in bed height maintained above 600° C. was also shown to vary with catalyst/diluent ratio. The minimum increase observed for all ratios tested was at least 13%, while the maximum was 22%. The results indicate that when exposed to higher temperature (e.g., greater than or equal to 600° C.), catalyst beds utilizing the engineered inert particles disclosed herein are more resilient in that the reactor bed height is not decreased when exposed to these temperature, as was seen with catalyst beds utilizing the T-64 system (i.e., unshaped particles).

Additionally, during the mixing of the diluents and reactor catalyst, it was observed that the T-64 grain grains tended to form areas of high concentration, or agglomerates, within the bulk of the reactor. The agglomeration of the diluents grains can cause a phenomenon called “channeling”, which occurs due to the tendency of a gas or liquid to flow through the path of least resistance. When this occurs, the path of least resistance is through the areas where the T-64 grains have agglomerated (see e.g., FIG. 3; arrows within circled areas). This preferential flow of the reactants through the agglomerates reduces the effective contact time with, and utilization of, the catalyst within the reactor bed. In addition, the preferential flow of the reactants through the agglomerates will create a local heat sink imbalance within the packed bed, leading to poor heat transfer efficiency. In contrast, agglomeration was not observed with the disclosed diluents (FIG. 2; arrows), and an even distribution throughout the bed was obtained after mixing with the catalyst material.

Finally, the disclosed diluent was observed to enhance heat transfer efficiency. Without wishing to be limited by theory, it is believed that the engineered shape of the diluent provided a greater surface area available for heat transfer, resulting in an increase in the heat transfer efficiency per unit volume. The irregular shape of the T-64 particle, in contrast, resulted in a reduction of the geometrical surface area available for heat transfer from the fluid to the solid phase and hence the heat transfer efficiency. This is consistent with the observed results, with the disclosed diluents outperforming the T-64 particle even though the T-64 particle has greater mass per unit volume.

The engineered inert particles, catalyst bed, and methods of making disclosed herein include at least the following embodiments:

### Embodiment 1

An engineered inert particle for use as a diluent in a catalyst bed in a fixed-bed reactor, comprising: an engineered inert particle comprising a cross-sectional shape having two opposing convex edges joined by and intersecting two opposing concave edges and a plurality of holes between said edges penetrating through the particle.

### Embodiment 2

The engineered inert particle of Claim 1, wherein the plurality is equal to three.

### Embodiment 3

The engineered inert particle of Claim 1 or Claim 2, comprising a central hole and two holes defining curved slots substantially parallel to the two convex edges.

### Embodiment 4

The engineered inert particle of any of Claims 1-3, wherein the two opposing convex edges have substantially the same radius of curvature.

### Embodiment 5

The engineered inert particle of any of Claims 1-3, wherein the two opposing convex edges have substantially the same radius of curvature.

### Embodiment 6

The engineered inert particle of any of Claims 1-5, wherein the particle has a length of greater than or equal to 6.4 mm.

### Embodiment 7

The engineered inert particle of any of Claims 1-5, wherein the particle has a length of less than or equal to 35 mm.

### Embodiment 8

A catalyst bed in a fixed-bed reactor for use in dehydrogenation of lower alkanes, comprising: (i) catalyst particles supporting a catalyst effective to promote said dehydrogenation; and (ii) engineered inert diluent particles comprising a cross-sectional shape having two opposing convex edges joined by and intersecting two opposing concave edges and a plurality of holes between said edges penetrating through the particle.

### Embodiment 9

The catalyst bed of Claim 8, wherein the plurality is equal to three.

### Embodiment 10

The catalyst bed of Claim 8 or Claim 9, comprising a central hole and two holes defining curved slots substantially parallel to the two convex edges.

### Embodiment 11

The catalyst bed of any of Claims 8-10, wherein the weight ratio of inert diluent particles to catalyst particles is greater than or equal to 1.

### Embodiment 12

The catalyst bed of Claim 11, wherein the weight ratio of inert diluent particles to catalyst particles is at least 65:35.

### Embodiment 13

The catalyst bed of any of Claims 8-12, wherein the catalyst particles are a different shape than the inert diluent particles.

### Embodiment 14

The catalyst bed of Claim 13, wherein the catalyst particles are cylindrical.

### Embodiment 15

The catalyst bed of any of Claims 8-14, wherein the diluent particles are large enough relative to the catalyst particles to facilitate the separation of the catalyst particles from the inert diluent particles, and the diluent particles from each other, in a packed bed.

### Embodiment 16

The catalyst bed of any of Claims 8-15, wherein the catalyst particles are cylinders x mm in diameter, and the inert diluent particles are larger than 2x mm in length.

### Embodiment 17

The catalyst bed of Claim 16, wherein the catalyst particles are cylinders 3.2 mm in diameter and 4.4 mm in height, and the inert diluent particles are larger than 6.4 mm in length.

### Embodiment 18

The catalyst bed of any of Claims 8-17, wherein each inert diluent particle has a length of greater than or equal to 6.4 mm.

### Embodiment 19

The catalyst bed of any of Claims 8-17, wherein each inert diluent particle has a length of less than or equal to 35 mm.

### Embodiment 20

The catalyst bed of any of Claims 8-19, wherein the fixed-bed reactor comprises, a hold-down layer, a catalyst layer comprising the inert diluent particles, and a support layer.

### Embodiment 21

A method of carrying out the dehydrogenation of lower alkanes in a fixed-bed reactor containing a catalyst bed, the catalyst bed comprising (i) catalyst particles supporting a catalyst effective to promote said dehydrogenation and (ii) engineered inert diluent particles, the method comprising: passing the lower alkane in gaseous form through the catalyst bed, wherein the engineered inert diluent particles have a cross-sectional shape having two opposing convex edges joined by and intersecting two opposing concave edges, and a plurality of holes between said edges penetrating through the particle.

### Embodiment 22

The method of Claim 21, wherein the plurality is equal to three.

### Embodiment 23

The method of Claim 21 or Claim 22, wherein said engineered inert diluent particles comprise a central hole and two holes defining curved slots substantially parallel to the two convex edges.

### Embodiment 24

The method of any of Claims 21-23, wherein the weight ratio of engineered inert diluent particles to catalyst particles in said catalyst bed is greater than or equal to 1.

### Embodiment 25

The method of Claim 24, wherein the weight ratio of engineered inert diluent particles to catalyst particles in said catalyst bed is at least 65:35.

### Embodiment 26

The method of any of Claims 21-25, wherein the catalyst particles are a different shape than the engineered inert diluent particles.

### Embodiment 27

The method of Claim 26, wherein the catalyst particles are cylindrical.

### Embodiment 28

The method of any of Claims 21-27, wherein the diluent particles are large enough relative to the catalyst particles to facilitate the separation of the catalyst particles from the inert diluent particles, and the diluent particles from each other, in a packed bed.

### Embodiment 29

The method of any of Claims 21-28, wherein the catalyst particles are cylinders x mm in diameter, and the inert diluent particles are larger than 2x mm in length.

### Embodiment 30

The method of Claim 29, wherein the catalyst particles are cylinders 3.2 mm in diameter and 4.4 mm in height, and the inert diluent particles are larger than 6.4 mm in length.

### Embodiment 31

The method of any of Claims 21-30, wherein each inert diluent particle has a length of greater than or equal to 6.4 mm.

### Embodiment 32

The method of any of Claims 21-30, wherein each inert diluent particle has a length of less than or equal to 35 mm.

The following terms, as used herein, have the meanings given below unless indicated otherwise. Terms and abbreviations not defined should be accorded their ordinary meaning as used in the art. Note also that singular articles, such as “a” and “an”, encompass the plural, unless otherwise specified or apparent from context.

A “catalyst particle” refers to an inert support particle, typically made of ceramic, glass or other inert material, comprising a catalyst, typically a metal or metal oxide catalyst, as used in heterogeneously catalyzed reactions, such as alkane dehydrogenation. Typically, the catalyst particle is porous, having a large surface area, with catalyst applied on the surface and within the pores.

An “inert diluents”, “inert particle”, or “inert packing” refers to a support particle used within a fixed bed reactor which contains essentially no catalyst and it is used for dilution of catalyst particles.

A “catalyst:diluent ratio”, “diluent:catalyst ratio”, or similar terminology, refers to the ratio, typically the weight ratio, of catalyst particles (not catalyst per se) to diluents particles (or vice versa) in a fixed bed or region of a fixed bed.

References to “surface area” of a particle, unless indicated otherwise, do not include the porosity of the material from which the particle is made.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention. The endpoints of all ranges directed to the same component or property are inclusive and independently combinable (e.g., ranges of “less than or equal to 25 wt %, or 5 wt % to 20 wt %,” is inclusive of the endpoints and all intermediate values of the ranges of “5 wt % to 25 wt %,” etc.). Disclosure of a narrower range or more specific group in addition to a broader range is not a disclaimer of the broader range or larger group. “Combination” is inclusive of blends, mixtures, alloys, reaction products, and the like. Furthermore, the terms “first,” “second,” and the like, herein do not denote any order, quantity, or importance, but rather are used to denote one element from another. The terms “a” and “an” and “the” herein do not denote a limitation of quantity, and are to be construed to cover both the singular and the plural, unless otherwise indicated herein or clearly contradicted by context. “Or” means “and/or.” The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

The modifier “about” used in connection with a quantity is inclusive of the stated value and has the meaning dictated by the context (e.g., includes the degree of error associated with measurement of the particular quantity). The notation “±10%” means that the indicated measurement can be from an amount that is minus 10% to an amount that is plus 10% of the stated value. The terms “front”, “back”, “bottom”, and/or “top” are used herein, unless otherwise noted, merely for convenience of description, and are not limited to any one position or spatial orientation. “Optional” or “optionally” means that the subsequently described event or circumstance can or cannot occur, and that the description includes instances where the event occurs and instances where it does not. Unless defined otherwise, technical and scientific terms used herein have the same meaning as is commonly understood by one of skill in the art to which this invention belongs. A “combination” is inclusive of blends, mixtures, alloys, reaction products, and the like.

All cited patents, patent applications, and other references are incorporated herein by reference in their entirety. However, if a term in the present application contradicts or conflicts with a term in the incorporated reference, the term from the present application takes precedence over the conflicting term from the incorporated reference

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. An engineered inert particle for use as a diluent in a catalyst bed in a fixed-bed reactor, comprising:
an engineered inert particle comprising a cross-sectional shape having two opposing convex edges joined by and intersecting two opposing concave edges and a plurality of holes between said edges penetrating through the particle.

2. The engineered inert particle of claim 1, wherein the plurality is equal to three.

3. The engineered inert particle of claim 1, comprising a central hole and two holes defining curved slots substantially parallel to the two convex edges.

4. The engineered inert particle of claim 1,
wherein the two opposing convex edges have substantially the same radius of curvature.

5. (canceled)

6. The engineered inert particle of claim 1, wherein the particle has a length of greater than or equal to 6.4 mm and less than or equal to 35 mm.

7. (canceled)

8. A catalyst bed in a fixed-bed reactor for use in dehydrogenation of lower alkanes, comprising:
(i) catalyst particles supporting a catalyst effective to promote said dehydrogenation; and
(ii) engineered inert diluent particles comprising a cross-sectional shape having two opposing convex edges joined by and intersecting two opposing concave edges and a plurality of holes between said edges penetrating through the particle.

9. The catalyst bed of claim 8, wherein the plurality is equal to three.

10. The catalyst bed of claim 8, comprising a central hole and two holes defining curved slots substantially parallel to the two convex edges.

11. (canceled)

12. The catalyst bed of claim 8, wherein the weight ratio of inert diluent particles to catalyst particles is at least 65:35.

13. The catalyst bed of claim 8, wherein the catalyst particles are a different shape than the inert diluent particles.

14. The catalyst bed of claim 13, wherein the catalyst particles are cylindrical.

15. The catalyst bed of claim 8, wherein the diluent particles are large enough relative to the catalyst particles to facilitate the separation of the catalyst particles from the inert diluent particles, and the diluent particles from each other, in a packed bed.

16. The catalyst bed of claim 8, wherein
the catalyst particles are cylinders x mm in diameter, and the inert diluent particles are larger than 2x mm in length.

17. The catalyst bed of claim 16, wherein the catalyst particles are cylinders 3.2 mm in diameter and 4.4 mm in height, and the inert diluent particles are larger than 6.4 mm in length.

18.-19. (canceled)

20. The catalyst bed of claim 8, wherein the fixed-bed reactor comprises, a hold-down layer, a catalyst layer comprising the inert diluent particles, and a support layer.

21. A method of carrying out the dehydrogenation of lower alkanes in a fixed-bed reactor containing a catalyst bed, the catalyst bed comprising (i) catalyst particles supporting a catalyst effective to promote said dehydrogenation and (ii) engineered inert diluent particles, the method comprising:
passing the lower alkane in gaseous form through the catalyst bed, wherein the engineered inert diluent particles have a cross-sectional shape having two opposing convex edges joined by and intersecting two opposing concave edges, and a plurality of holes between said edges penetrating through the particle.

22.-24. (canceled)

25. The method of claim 21, wherein the weight ratio of engineered inert diluent particles to catalyst particles in said catalyst bed is at least 65:35.

26. The method of claim 21, wherein the catalyst particles are a different shape than the engineered inert diluent particles.

27. (canceled)

28. The method of claim 21, wherein the diluent particles are large enough relative to the catalyst particles to facilitate the separation of the catalyst particles from the inert diluent particles, and the diluent particles from each other, in a packed bed.

29. The method of claim 21, wherein the catalyst particles are cylinders x mm in diameter, and the inert diluent particles are larger than 2x mm in length.

30.-32. (canceled)
